(19) United States
(12) Patent Application Publication
(10) Pub. No.: US 2006/0111332 A1
(43) Pub. Date: May 25, 2006

(54) Process and intermediates to prepare17beta-hydroxy-7alpha-methyl-19-nor-17alpha-pregn -5(10)-en-20-yn-3-one

(75) Inventors: Jacek Martynow, Warszawa (PL); Andrzej Kutner, Warszawa (PL); Wieslaw Szelejewski, Warszawa (PL); Wanda Wojciechowska, Warszawa (PL); Hanna Fitak, Warszawa (PL)

Correspondence Address: WELSH & KATZ, LTD, 120 S RIVERSIDE PLAZA, 22ND FLOOR, CHICAGO, IL, 60606, US

(73) Assignee: Institut Farmaceutyczny, Warszawa (PL)

(21) Appl. No.: 10/530,139

(22) PCT Filed: Oct. 1, 2003
(86) PCT No.: PCT/PL03/00099

§ 371 Date: Oct. 11, 2005

(30) Foreign Application Priority Data
Oct. 4, 2002 (PL) P356465

(51) Int. Cl. A61K 31/56; C07J 7/00

(52) U.S. Cl. 514/182; 552/558

(57) ABSTRACT

The present invention is a process for the preparation of 17β-hydroxy-7α-methyl-19-nor-17α-pregn-5(10)-en-20-yn-3-one (17α-ethynyl-17β-hydroxy-7α-methyl-5(10)-estren-3-one, tibolone) of formula 1, which comprises hydrolysis of 17α-ethynyl-17β-hydroxy-7α-methyl-5(10)-estrene 3,3-cyclic ketals of formula 2, where groups R₁, R₂, R₃ and R₄ are hydrogen atoms or alkyl groups, or R₁ and R₃, taken together with the carbon atoms within the dioxolane ring to which they are attached, form an alicyclic ring fused to the dioxolane ring, with R₂ and R₄ being hydrogen atoms, or R₁ and R₃ together with the carbon atoms to which they are attached form an aromatic ring fused to the dioxolane ring, where R₂ and R₄, taken together, form a chemical bond within said aromatic ring. In addition, the present invention includes an intermediate, compound of formula 2 and two processes to prepare 17α-ethynyl-17β-hydroxy-7α-methyl-5(10)-estrene 3,3-cyclic ketals of formula 2: (a) by contacting 17α-ethynyl-17β-hydroxy-7α-methyl-4-estren-3-one with vicinal diols in the presence of a protic acid, and (b) by contacting 7α-methyl-5(10)-estrene-17-one 3,3-cyclic ketals of formula 4, where R₁-R₄ are defined as above, with metal acetylides, in inert solvents.

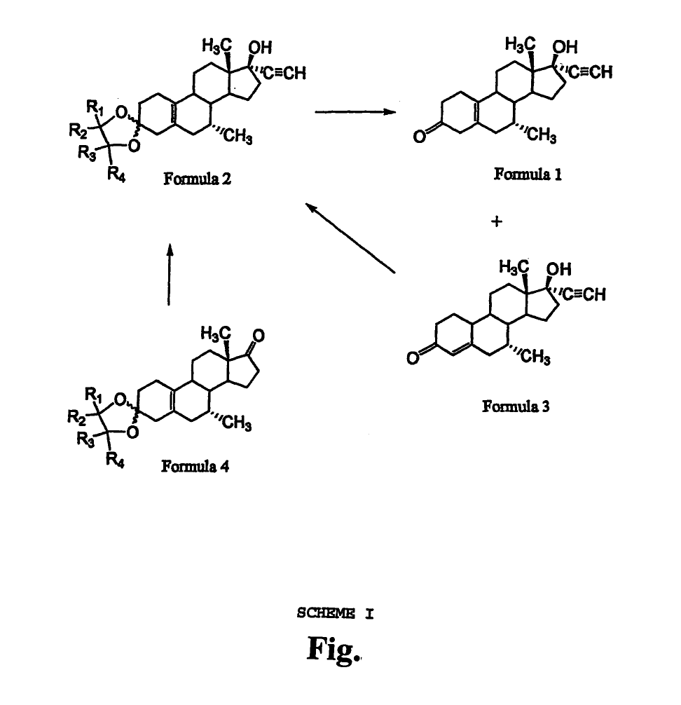

### CROSS REFERENCES TO RELATED APPLICATION

This application is a national phase application based upon priority International PCT Patent Application No. PCT/PL2003/000099 filed Oct. 1, 2003, International Publication No. WO 2004/031204 A2 published Apr. 15, 2004, which is based upon priority Polish Application P35465 filed Oct. 4, 2002.

### BACKGROUND OF THE INVENTION

1. Field of the Invention

The present invention is a process, including intermediates, to produce tibolone (17β-hydroxy-7α-methyl-19-nor-17α-pregn-5(10)-en-20-yn-3-one; 17α-ethynyl-17β-hydroxy-7α-methyl-5(10)-estren-3-one), a pharmaceutical agent useful in treating postmenopausal conditions and for the prevention of osteoporosis.

2. Description of the Related Art

Dutch patent NL 6,406,797 discloses tibolone and a process for its preparation which comprises hydrolysis of the enol ether grouping present in 17α-ethynyl-17β-hydroxy-3-methoxy-7α-methyl-2,5(10)-estradiene. 17α-Ethynyl-17β-hydroxy-3-methoxy-7α-methyl-2,5(10)-estradiene was prepared in three synthetic steps from 17β-hydroxy-3-methoxy-7α-methyl-1,3,5(10)-estratriene on the way of a Birch reduction to the 2,5(10)-diene, followed by an Oppenauer oxidation at C(17) and an acetylide addition to the C(17)-carbonyl.

Helvetica Chim. Acta 50, 1453 (1967) describes a reaction sequence leading from 17β-hydroxy-3-methoxy-7α-methyl-1,3,5(10)-estratriene to 17α-ethynyl-17β-hydroxy-3-methoxy-7α-methyl-2,5(10)-estradiene which was then hydrolyzed to tibolone.

J. Am. Chem. Soc. 86, 742 (1964) and Helvetica Chim. Acta 50, 289 (1967) disclose the preparation of 17β-hydroxy-3-methoxy-7α-methyl-1,3,5(10)-estratriene from readily available testosterone 17-esters in ca. eight synthetic steps.

Tetrahedron Lett. 38, 7997 (1997) and Italian patent application IT 2000MI0918 A1 both describe the preparation of 17β-hydroxy-3-methoxy-7α-methyl-1,3,5(10)-estratriene in two steps from 17β-tetrahydropyranyloxy-3-methoxy-1,3,5(10)-estratrien-6-one, which is readily derived from β-estradiol in four synthetic steps.

Italian patent application IT 99MI2128 A1 describes the route to tibolone via the 3,3-dimethoxy derivative, 3,3-dimethoxy-17αethynyl-17β-hydroxy-7α-methyl-5(10)-estrene, which is obtained in 6 steps from 17β-hydroxy-3-methoxy-7α-methyl-1,3,5(10)-estratriene.

International Patent Application PCT/EP/99/07768 discloses a process for high purity, highly stable tibolone preparation which comprises hydrolysis of the 3,3-dimethylketal grouping present in 3,3-dimethoxy-17α-ethynyl-17β-hydroxy-7α-methyl-5(10)-estrene. 3,3-Dimethoxy-17α-ethynyl-17α-hydroxy-7α-methyl-5(10)-estrene was the only 3,3-ketal used and claimed as a substrate for the hydrolysis reaction by which tibolone was prepared.

Recl. Trav. Chim. Pays-Bas 105, 111 (1986) discloses a process for tibolone preparation which comprises hydrolysis of the 3,3-dimethylketal grouping present in 3,3-dimethoxy-17α-ethynyl-17β-hydroxy-7α-methyl-5(10)-estrene. 3,3-Dimethoxy-17α-ethynyl-17β-hydroxy-7α-methyl-5(10)-estrene was the only 3,3-ketal used as a substrate for the hydrolysis reaction by which tibolone was prepared. 3,3-Dimethoxy-17α-ethynyl-17β-hydroxy-7α-methyl-5(10)-estrene was prepared in eight synthetic steps from 17β,19-diacetoxy-4,6-androstadien-3-one. A four-step preparation of 17β,19-diacetoxy-4,6-androstadien-3-one from readily available 3β,17β-diacetoxy-5-androstene is disclosed in Experientia 18, 464 (1962) and in Belgian patent BE 620,225.

A process to prepare 7α-methyl-19-oxo-4-androst-3,17-dione in 4 steps from 17β,19-diacetoxy-4,6-androstadien-3-one (or in eight steps from the readily available 3β,17β-diacetoxy-5-androstene) is also disclosed in Recl. Trav. Chim. Pays-Bas 105, 111 (1986).

U.S. Pat. No. 3,475,465 discloses a one step preparation of tibolone from 7-methyl-19-oxo-4-androst-3,17-dione in the presence of potassium metal and acetylene in liquid ammonia, albeit the yield was not specified and for a closely related compound the yield was below 50%.

U.S. Pat. No. 3,928,398 discloses a process to prepare 17β-hydroxy-7α-methyl-4-estren-3-one from 19-nortestosterone in four steps.

J. Med. Chem. 35, 2113 (1992) describes the preparation of 3,3-ethylenedioxy-7α-methyl-5(10)-estren-17-one in two synthetic steps from 17β-hydroxy-7α-methyl-4-estren-3-one. The conditions to obtain 3,3-ethylenedioxy-17β-hydroxy-7α-methyl-5(10)-estrene in one step from 17β-hydroxy-7α-methyl-4-estren-3-one and ethylene glycol are also described. This publication also discloses a highly efficient hydrolysis reaction of 3,3-ethylenedioxy-15α-hydroxy-7α-methyl-5(10)-estren-17-one to 15α-hydroxy-7α-methyl-4-estren-3,17-dione, under the conditions of HCl in MeOH.

Synthesis 501 (1981) reviews examples of acid catalyzed ketal (acetal) preparation from carbonyl compounds and alcohols, including 1,2-diols.

J. Org. Chem. 54, 5180 (1989) describes a preparation of 3,3-ethylenedioxy-17α-ethynyl-17β-hydroxy-5-androstene from 17α-ethynyl-17β-hydroxy-4-androsten-3-one (ethisterone) and ethylene glycol, in the presence of p-toluenesulfonic acid and trimethyl orthoformate.

German Patent DE 3,337,179 describes the preparation of a mixture of 3,3-ethylenedioxy-17α-ethynyl-17β-hydroxy-5-estrene and 3,3-ethylenedioxy-17α-ethynyl-17β-hydroxy-5(10)-estrene by contacting 17α-ethynyl-17β-hydroxy-4-estren-3-one with ethylene glycol, in the presence of trimethyl orthoformate and p-toluenesulfonic acid, in a dichloromethane solution.

U.S. Pat. No. 3,904,611 discloses the preparation of 17β-acetoxy-3,3-ethylenedioxy-5(10)-estrene by reaction of 17β-acetoxy-4-estren-3-one with ethylene glycol, in the presence of p-toluenesulfonic acid, under reflux for 16 hours. 17β-Acetoxy-3,3-ethylenedioxy-5(10)-estrene was then hydrolyzed in the presence of malonic acid, in an acetone-water mixture.

Synthetic Commun. 27, 2197 (1997) addresses the issue of the selectivity observed in the reaction of 19-norsteroidal 4-en-3-ones with ethylene glycol, in the presence of various catalysts. The ratio of 3,3-ethylenedioxy-5,6-elkenes to 3,3-ethylenedioxy-5(10)-elkenes varied from 50:50 to 0:100, depending on the catalyst.

Recl. Trav. Chim. Pays-Bas 92, 1047 (1973) addresses the issue of the selectivity observed in the reaction of steroidal 4-en-3-ones with ethylene glycol, catalyzed by various acids. Importantly, the formation of steroidal 4,5-unsaturated 3,3-ethylenedioxy ketals versus 5,6-unsaturated 3,3-ethylenedioxy ketals was correlated with pKa values of the acids. 4,5-Unsaturated 3,3-ethylenedioxy products were obtained exclusively in cases when protic acids with pKa value above 3 were used. 5,6-Unsaturated 3,3-ethylenedioxy products were obtained exclusively in cases when a protic acid with pKa value less than ca. 1 was used.

Steroids 60, 414 (1995) reports on the selectivity observed in the reaction of 19-norsteroidal 4-en-3-ones with ethylene glycol, catalyzed by an acid. Accordingly, the ratio of the 5,6-unsaturated 3,3-ethylenedioxy product versus the 5(10)-unsaturated 3,3-ethylenedioxy product is dependent on reaction time, temperature and acid concentration, such that less vigorous conditions favor the formation of the 5,6-alkene.

U.S. Pat. No. 4,308,265 discloses a process to prepare 17α-ethynyl-17β-hydroxy-7α-methyl-4-estren-3-one (7α-methylnorethindrone) and its esters. Thus, 17α-ethynyl-17β-hydroxy-3-methoxy-7α-methyl-2,5(10)-estradiene was prepared from 3-methoxy-7α-methyl-1,3,5(10)-estratrien-17-one. U.S. Pat. No. 4,308,265 discloses also that 3,3-ethylenedioxy-7α-methyl-5(10)-estren-17-one was ethynylated at C(17) and a 17-ethynyl-17-hydroxy compound thus obtained was then hydrolyzed with dilute hydrochloric acid to a crystalline enone, which was esterified by heptanoic anhydride to yield 7α-methylnorethindrone enanthate, which is a steroidal 4-en-3-one, and not a 5(10)-en-3-one. The structures of the intermediates in this route to 7α-methylnorethindrone enanthate were not supported by any physicochemical or other data. Also, no experimental details were given for the ketalization step. In light of the prior art cited above regarding the various positional isomers of alkenes which may form upon ketalization of 4-en-3-ones, when the conditions are not carefully controlled, the alternative structures of 3,3-ethylenedioxy-7α-methyl-5-estren-17-one or 3,3-ethylenedioxy-7α-methyl-4-estren-17-one are very likely as the intermediates on the way to 7α-methylnorethindrone esters as described in U.S. Pat. No. 4,308,265, especially that a purification of the ketal species is not described. Also, patent application U.S. Pat. No. 4,308,265 does not give any indication that the hydrolysis of 3,3-ethylenedioxy-17α-ethynyl-17-hydroxy-7α-methyl-5(10)-estrene in the presence of an acid may lead to 3-keto-5(10-estrene derivatives.

East Germany Patent DD 143,781 describes efficient oxidation of 17β-hydroxy-3,3-dimethoxy steroids to 3,3-dimethoxy-17-ketosteroids under the conditions of pyridinium chlorochromate and sodium acetate in dichloromethane.

European Patent Application EP 0 700 926 A1 discloses a process for the preparation of gestodene. Disclosed is an Oppenauer oxidation of a mixture of 3,3-ethylenedioxy-17β-hydroxy-18-methyl-5-estrene and 3,3-ethylenedioxy-17β-hydroxy-18-methyl-5(10)-estrene to a mixture of 3,3-ethylenedioxy-18-methyl-5-estren-17-one and 3,3-ethylenedioxy-18-methyl-5-estren-17-one. In the final step of gestodene synthesis, a mixture of a 3,3-ethylenedioxy-5-ene and a 3,3-ethylenedioxy-5(10)-ene is hydrolyzed in the presence of acid, affording exclusively gestodene, which is a 19-norsteroidal 4-en-3-one.

U.S. Pat. Nos. 3,318,928 and 4,874,754 both give examples of the reaction of steroidal 17-ketones with metal acetylides, leading to 17α-ethynyl-17-hydroxy derivatives.

U.S. Pat. No. 2,806,030 discloses a process for the preparation of 17α-ethynyl-19-nortestosterone. Thus, 3,3-ethylenedioxy-5(10)-estren-17-one in the presence of potassium alkoxide and acetylene afforded 3,3-ethylenedioxy-17α-ethynyl-17β-hydroxy-5(10)-estrene. 3,3-Ethylenedioxy-17α-ethynyl-17β-hydroxy-5(10)-estrene was hydrolyzed in acidic medium to 17α-ethynyl-19-nortestosterone, which is a 19-norsteroidal 4-en-3-one.

UK Patent Application GB 2,185,257 A describes a mild hydrolysis of 17β-acetoxy-3,3-ethylenedioxy-6β-methyl-5(10)-estrene, which in the presence of acetic acid, water and diethyl ether afforded 17β-acetoxy-3,3-methyl-5(10)-estren-3-one.

J. Org. Chem. 43, 1821 (1978) disclosed a general procedure for the hydrolysis of β,γ-unsaturated ketals, under the conditions of 80% aqueous acetic acid.

Synthetic Commun. 25, 395 (1995) describes a method for the cleavage of ketals (acetals) using CuSO₄ adsorbed on silica gel. Two examples of steroidal 3,3-ethylenedioxy-5-enes are given. Each of these ketals, when treated with CuSO₄ adsorbed on silica gel in a chloroform solution, afforded respective steroidal 4-en-3-one as the only products. No β,γ-unsaturated ketones formed from the 3,3-ethylenedioxy-5-enes.

### BRIEF SUMMARY OF INVENTION

Disclosed is a process for the preparation of 17β-hydroxy-7α-methyl-19-nor-17α-pregn-5(10)-en-20-yn-3-one of formula 1, which comprises:
(I) hydrolysis of 17α-ethynyl-17β-hydroxy-7-methyl-5(10)-estrene 3,3-cyclic ketals of formula 2, where:
   (1) each of R₁, R₂, R₃ and R₄ is a hydrogen atom or a C₁₋₄alkyl group, or
   (2) R₁ and R₃ are taken together to form an alicyclic ring together with the carbon atoms in the dioxolane ring to which the groups are attached and R₂, R₄ are hydrogen atoms, or
   (3) R₁ and R₃ are taken together to form an aromatic ring together with the carbon atoms in the dioxolane ring to which they are attached, and R₂, R₄ are taken together to form a chemical bond participating in the aromatic electron system of the aromatic ring formed by R₁ and R₃, in the presence of salts of transition metals, salts of lithium or salts of magnesium, and
   (b) separating 17β-hydroxy-7β-methyl-19-nor-17α-pregn-5(10)-en-20-yn-3-one obtained in step (a) from 17β-hydroxy-7α-methyl-19-nor-17α-pregn-4-en-20-yn-3-one by-product of formula 3; and
   (c) converting 17β-hydroxy-7α-methyl-19-nor-17α-pregn-4-en-20-yn-3-one obtained as a by-product in step (b) to the ketal of formula 2, wherein R₁-R₄ are defined as above, which is then hydrolyzed to 17β-hydroxy-7α-methyl-19-nor-17α-pregn-5(10)-en-20-yn-3-one in step (a).

A more detailed description of the invention is provided in the following description and appended claims taken in conjunction with the accompanying drawing.

### BRIEF DESCRIPTION OF THE DRAWING

The drawing is a chart of a process for tibolone synthesis by hydrolysis of 3,3-ketals of formula 2 and illustrating chemical formulas 1, 2, 3 and 4.

### DETAILED DESCRIPTION OF THE INVENTION

The following is a detailed description and explanation of the preferred embodiments and best modes for embodying the invention along with some examples thereof.

In a majority of the processes for tibolone synthesis disclosed to date, 17-hydroxy-3-methoxy-7α-methyl-1,3,5(10)-estratriene is the key intermediate. The process presented in Dutch Patent NL 6406797 requires that 17β-hydroxy-3-methoxy-7α-methyl-1,3,5(10)-estratriene be reduced under the Birch reduction conditions, then the 17β-hydroxy group is oxidized under the Oppenauer oxidation conditions, followed by an acetylide addition to the 17-ketone, which results in 17α-ethynyl-17β-hydroxy-3-methoxy-7α-methyl-2,5(10)-estradiene. This compound is subsequently hydrolyzed under mild acidic conditions, leading to tibolone. In alternative, though related processes (e.g. van Vliet at al. Recl. Trav. Chim. Pays-Bas 105, 111 (1986); patent application IT 99MI2128 A1) the 3-keto group is initially protected in the form of a 3,3-dimethylacetal, then the acetylide addition at C(17) is carried out and, finally, the thus obtained 3,3-dimethoxy-17α-ethynyl-17β-hydroxy-7α-methyl-5(10)-estrene is hydrolyzed to tibolone. The deprotection of the unstable dimethylacetal, under very weakly acidic conditions, resulted exclusively or almost exclusively in 17α-ethynyl-17β-hydroxy-7α-methyl-5(10)-estren-3-one, while the application of stronger acids led to the 4-ene isomer, 17α-ethynyl-17β-hydroxy-7α-methyl-4-estren-3-one. The latter compound, which is a conjugated ketone, often is a ubiquitous impurity of tibolone.

All the disclosed processes for tibolone synthesis which make use of 17β-hydroxy-3-methoxy-7α-methyl-1,3,5(10)-estratriene require that the aromatic ring A in this compound be reduced under the Birch conditions (March, J. Advanced Organic Chemistry. Reactions, Mechanisms and Structure. 4^{th} Ed.; John Wiley and Sons; New York, N.Y., 1992; p. 781). This reduction method, however, poses technical difficulties and environmental hazards due to the need for a large excess of liquid ammonia, and due to the use of pyrophoric metals, such as sodium or lithium. Similarly, the synthesis of the key intermediate, 17β-hydroxy-3-methoxy-7α-methyl-1,3,5(10)-estratriene, is often very problematic because the required starting materials are expensive and/or are not easily accessible. Also, the known conditions necessary for the synthesis of 17β-hydroxy-3-methoxy-7α-methyl-1,3,5(10)-estratriene are often troublesome, such as the low temperature LIDAKOR reaction or a step involving large amounts of boron-derived side-products (Tedesco, R. Et al. Tetrahedron Lett. 38, 7997 (1997); patent application IT 2000MI0918 A1).

These difficulties are, in part, avoided in the processes for tibolone synthesis in which 6-dehydro-19-hydroxytestosterone derivatives are used as the substrate, instead of 17β-hydroxy-3-methoxy-7αmethyl-1,3,5(10)-estratriene (van Vliet et al. Recl. Trav. Chim. Pays-Bas 105, 111 (1986); U.S. Pat. No. 3,475,465). However, according to prior art such 19-oxygenated compounds are not easily accessible, either.

All of the known processes for tibolone synthesis require during the last step of the synthesis that a hydrolysis of a 3-alkoxy-2,5(10)-diene or a 3,3-dimethoxy acetal group be carried out, and, importantly, other 3,3-ketals (acetals) have not been disclosed to date as substrates for tibolone.

Thus, the number of existing methods suitable for a short, large scale synthesis of tibolone from commercially available steroids is very limited. The shortest routes disclosed to date are: (a) the route via the 3-methoxy-2-ene derivative which is obtained from 17β-hydroxy-3-methoxy-7α-methyl-1,3,5(10)-estratriene, which is derived from β-estradiol [NL 6,406,797 and Tetrahedron Lett. 38, 7997 (1997); a ten step route] or is derived from testosterone [J. Am. Chem. Soc. 86, 742 (1964) and Helvetica Chim. Acta 50, 289 (1967); ca. thirteen step route], (b) the route to tibolone via the 3,3-dimethoxy derivative, which is obtained in 6 steps from 17β-hydroxy-3-methoxy-7α-methyl-1,3,5(10)-estratriene (Italian patent application IT 99MI2128 A1), and (c) the route in nine steps from 3β,17β-diacetoxy-5-androstene via 7α-methyl-19-oxo-4-androst-3,17-dione [U.S. Pat. No. 3,475,465; Recl. Trav. Chim. Pays-Bas 105, 111 (1986); Experientia 18, 464 (1962)].

These processes are troublesome due to: (a) the need to carry out the technologically difficult Birch reduction of 17β-hydroxy-3-methoxy-7α-methyl-1,3,5(10)-estratriene (requires large amounts of pyrophoric metals and liquid ammonia) and (b) modest overall yields and the need for laborious chromatographic separations of 7-methylsteroid intermediates isomeric at C(7). Again, the 3,3-dimethoxy (3,3-dimethyl ketal) derivative is the only type of a steroidal 3,3-ketal used for the direct hydrolysis to tibolone—none of the existing processes for tibolone preparation comprises a hydrolysis step of other steroidal 3,3-ketal derivatives. Similarly, none of the existing methodologies for tibolone synthesis has taken advantage of the deconjugative ketalization reaction in order to form the 5,(10)-double bond present in tibolone.

Unexpectedly, it has now been found that according to the present invention tibolone can be prepared in high yield on the way of a one step process comprising the hydrolysis of 17α-ethynyl-17β-hydroxy-7α-methyl-5(10)-estrene 3,3-cyclic ketals of formula 2, where:
(1) each of R₁, R₂, R₃ and R₄ is a hydrogen atom or a C₁₋₄ alkyl group, or
(2) R₁ and R₃ are taken together to form an alicyclic ring together with the carbon atoms in the dioxolane ring to which the groups are attached and R₂, R₄ are hydrogen atoms, or
(3) R₁ and R₃ are taken together to form an aromatic ring together with the carbon atoms in the dioxolane ring to which they are attached, and R₂, R₄ are taken together to form a chemical bond participating in the aromatic electron system of the aromatic ring formed by R₁ and R₃.
   in the presence of salts of transition metals, salts of lithium or salts of magnesium;
(b) separating 17β-hydroxy-7α-methyl-19-nor-17α-pregn-5(10)-en-20-yn-3-one obtained in step (a) from 17β-hydroxy-7α-methyl-19-nor-17α-pregn-4-en-20-yn-3-one by-product of formula 3; and
(c) converting 17β-hydroxy-7α-methyl-19-nor-17α-pregn-4-en-20-yn-3-one obtained as a by-product in step (b) to the ketal of formula 2, wherein R₁-R₄ are defined as above, which is then hydrolyzed to 17β-hydroxy-7α-methyl-19-nor-17α-pregn-5(10)-en-20-yn-3-one in step (a).

This finding of the present invention is even more surprising in light of the reported process for the synthesis of a derivative of 17α-ethynyl-17β-hydroxy-7α-methyl-4-estren-3-one (7α-methylnorethindrone) from a 3,3-ethylenedioxy-5(10)-ene, -4-ene and/or -5-ene precursors (Blye, R. Et al., U.S. Pat. No. 4,308,265). These authors found that the hydrolysis of the 3,3-ethylenedioxy acetal carried out under acidic conditions gave exclusively 7α-methylnorethindrone, which is a conjugated ketone possessing a 4-en-3-one structure, and not the 5(10)-en-3-one structure, which is found in tibolone.

The 3,3-cyclic ketals of formula 2 have never been reported as substrates for a one-step preparation of tibolone. Prior art also includes other reports on the synthesis of steroidal 4-en-3-ones from 3,3-ethylenedioxy-5(10)-ene or -5-ene precursors [EP 0 700 926 A1; U.S. Pat No. 2,806,030; Synth. Commun. 25, 395 (1995)], including a report which addresses the hydrolysis of 3,3-ethylenedioxy-15α-hydroxy-7αmethyl-5(10)-estren-17-one, which, in the presence of hydrochloric acid, afforded 15α-hydroxy-7α-methyl-4-estren-3,17-dione [J. Med. Chem. 35, 2113, (1992)]. Other types of acidic conditions used for the hydrolysis of steroidal ethylenedioxy ketals, which were not 7-methyl-5(10)-estrene derivatives, include aqueous acetic acid, aqueous acetic acid/Et₂O or malonic acid/acetone-water [U.S. Pat. No. 3,904,611; GB 2,185,257A; J. Org. Chem. 43, 1821 (1978)]. The presence of the 7α-methyl group is known to influence the chemistry of estrane derivatives to a large degree [J. Med. Chem. 35, 2113 (1992) and Steroids 60, 414 (1995)].

Equally unexpectedly, a process for the preparation of structurally defined 17α-ethynyl-17β-hydroxy-7α-methyl-5(10)-estrene 3,3-cyclic ketals of formula 2 has not been put forth to date. Example 3 of U.S. Pat. No. 4,308,265 discloses that 3,3-ethylenedioxy-7α-methyl-5(10)-estren-17-one was ethynylated at C(17) and a 17-ethynyl-17-hydroxy compound thus obtained was then hydrolyzed with dilute hydrochloric acid to a crystalline enone, which was esterified by heptanoic anhydride to yield 7α-methylnorethindrone enanthate, which is a steroidal 4-en-3-one, and not a 5(10)-en-3-one. The structures of the intermediates in this route to 7α-methylnorethindrone enanthate were not supported by any physicochemical or other data. Also, no experimental details were given for the crucial ketalization step and no purification of the product was described. Incidentally, the prior art regarding the various positional isomers of alkenes which may form upon ketalization of 4-en-3-ones [J. Med. Chem. 35, 2113 (1992) and Steroids 60, 414 (1995); Synthetic Commun. 27, 2197 (1997); Recl. Trav. Chim. Pays-Bas 92, 1047 (1973)] teaches that, when the conditions are not carefully controlled, the alternative structures of 3,3-ethylenedioxy-7α-methyl-5-estren-17-one or 3,3-ethylenedioxy-7α-methyl-4-estren-17-one are very likely on the way to 7α-methylnorethindrone esters as described in U.S. Pat. No. 4,308,265. Importantly, in the patent U.S. Pat. No. 4,308,265 there is no indication that the hydrolysis of 3,3-ethylenedioxy-17βethynyl-17-hydroxy-7α-methyl-5(10)-estrene in the presence of acid may lead to tibolone.

It has now been found that, according to the present invention, chemically pure and structurally defined 17α-ethynyl-17β-hydroxy-7α-methyl-5(10)-estrene 3,3-cyclic ketals of formula 2 can be prepared in one step from 7α-methyl-5(10)-estren-17-one. 3,3-cyclic ketals of formula 4, where R₁-R₄ are as defined above, by reacting a compound of formula 4 with metal acetylides in inert solvents while maintaining the reaction mixture temperature in the range from about −50° C. to about +30° C., followed by a purification procedure, preferably by crystallization, more preferably by crystallization from a mixture of solvents containing 0%-50% THF, 0%-50% 1,4-dioxane, 0%-50% toluene and 0%-100% of ethyl acetate, and most preferably by crystallization from ethyl acetate, which is found to be particularly efficient in removing any positional alkene isomers from the 5(10)-alkene product.

Another unexpected finding of the present invention is a process for the preparation of 17α-ethynyl-17β-hydroxy-7α-methyl-5(10)-estrene 3,3-cyclic ketals of formula 2, where R₁-R₄ are as defined above, in one step from 17α-ethynyl-17β-hydroxy-7α-methyl-4-estren-3-one and vicinal diols, in the presence of a protic acid, preferably in the presence of a dehydrating agent and a hydrocarbon co-solvent, most preferably in the presence of a protic acid with pKa less than ca. 1.5, a trialkyl orthoformate chosen from the group comprising trimethyl orthoformate, triethyl orthoformate, triisopropyl orthoformate, and, optionally, a co-solvent chosen from the group comprising toluene or xylenes.

The process of the present invention allows for an efficient preparation of tibolone (formula 1, Chart) from 3,3-ethylenedioxy-17α-ethynyl-17β-hydroxy-7α-methyl-5(10)-estrene or from other ketals of the present invention, described by formula 2. The practical value of this finding is best reflected by the fact that, in combination with the known synthesis of the compounds of formula 4 [e.g. U.S. Pat. No. 3,928,398; J. Med. Chem. 35, 2113 (1992)], tibolone can now be obtained in seven synthetic steps from the commercially available 19-nortestosterone.

The substrates necessary to accomplish the synthesis of tibolone according to the process of the present invention, are easily available. It has been reported that 7α-methyl-5(10)-estren-17-one 3,3-cyclic ketals of formula 4 can be prepared in two synthetic steps from 7α-methyl-19-nortestosterone [R₁-R₄=H; J. Med. Chem. 35, 2113 (1992)]. The first step of this preparation is a deconjugative ketalization process comprising a reaction of 7α-methyl-19-nortestosterone with ethylene glycol in the presence of p-toluenesulfonic acid, affording 3,3-ethylenedioxy-17β-hydroxy-7α-methyl-5(10)-estrene. However, other vicinal diols are also known to react with 3-keto steroids [Synthesis 501 (1981)] affording 3,3-ketals of a dioxolane structure where the dioxolane ring is substituted with one or more C₁₋₄ alkyl group(s) or the dioxolane ring is condensed with an alicyclic or an aromatic ring.

The second step of the preparation of 7α-methyl-5(10)-estren-17-one 3,3-cyclic ketals of formula 4 [R₁-R₄=H; J. Med. Chem. 35, 2113 (1992)] is an unbuffered PCC oxidation of 3,3-ethylenedioxy-17β-hydroxy-7β-methyl-5(10)-estrene. Many alternative methods for mild oxidation of 17-hydroxy steroids have also been disclosed (e.g. in patents DE 3,337,179 and EP 0 700 926 A1).

A process for the preparation of 7α-methyl-19-nortestosterone in four technological steps and in a good chemical yield from 19-nortestosterone has been put forth in U.S. Pat. No. 3,928,398. However, it may readily be apparent to those skilled in the art that the last two steps of this 7α-methyl-19-nortestosterone preparation (1,6-conjugate methylation followed by double bond isomerisation with concomitant cleavage of the 17-acetate to 17-hydroxyl) can be performed in one reaction vessel, thus shortening the route from 19-nortestosterone to 7α-methyl-19-nortestosterone to three technological steps: (a) nortestosterone enolization-peracetylation to 3,17β-diacetoxy-3,5-estradiene, (b) bromination-dehydrobromination to 17β-acetoxy-4,6-estradien-3-on and (c) 1,6-conjugate methylation (performed e.g. with Me₂CuLi) followed by double bond isomerisation with concomitant cleavage of the 17-acetate to 17-hydroxyl (performed e.g. by the addition to the reaction mixture of a KOH/MeOH solution) affording 7α-methyl-19-nortestosterone. The purification of 7α-methyl-19-nortestosterone from the 7β-methyl isomer is easily accomplished by crystallization.

The novel process for tibolone synthesis according to the present invention by hydrolysis of 3,3-ketals of formula 2, is presented in the Scheme I. It has now been found that the choice of the appropriate conditions for the hydrolysis reaction according to the process of the present invention is crucial to the successful synthesis of the desired product. According to the present invention, the reaction can be carried out in an organic solvent, optionally in the presence of water, and is carried out under the conditions chosen from two alternative types of conditions according to the process of the present invention, facilitating the hydrolysis reaction, which are listed below:

(a) under the conditions of the first type, the hydrolysis reaction is carried out in the presence of an acid, preferably an organic acid of medium strength (pKa/_{H2O}=2-5). Appropriate acids are chosen from the group including, but not limited to, oxalic acid, acetic acid, fumaric acid, formic acid, malonic acid and pyridinium p-toluenesulfonate. Most preferred is formic acid, or

(b) under the conditions of the second type, the hydrolysis reaction is carried out in the presence of a transition metal salt or a salt of lithium or magnesium, preferably a salt of lithium, iron, magnesium or copper. Preferred salts are copper(II) sulfate, copper(II) chloride, iron(III) chloride, lithium(I) tetrafluoroborate or magnesium(II) trifluoroacetate. Most preferred is copper(II) sulfate.

According to the present invention, the hydrolysis reaction is carried out in a mixture of solvents consisting of 0%-99% water and 0%-100% of an organic solvent selected from a group including, but not limited to: THF, CHCl₃, 1,4-dioxane, CH₂CL₂, acetone, acetonitrile, ethylmethyl ketone, diethyl ketone, 1,3-dioxolane, 1,2-dimethoxyethane, 1,2-diethoxyethane, and 0%-100% of a C₁₋₄ alcohol.

The hydrolysis reaction according to the process of the present invention can be carried out at a broad range of temperatures from 0° C. to 200° C., more preferably 15° C.-150° C. and most preferably 30° C.-90° C. The progress of the hydrolysis reaction may be monitored by analytical methods, preferably by HPLC or TLC on a “reversed phase” such as a C-18 phase. The reaction time should be sufficiently long to allow for a complete conversion of the substrate ketal of formula 2, and not for a substantially longer time. This is important, since after a longer reaction time, the formation of the desired tibolone of formula 1 is accompanied by the formation of increasing amounts of 17α-ethynyl-17β-hydroxy-7α-methyl-4-estren-3-one (7α-methylnorethisterone) of formula 3.

It has now been found that in order to ensure a good purity of tibolone prepared by the hydrolysis reaction according to the present invention, the ketal (acetal) substrate of formula 2 must be of purity better than 90%—if this condition is not fulfilled difficulties with purification may offset the benefits of this short synthetic route to tibolone.

According to the present invention, after the usual work-up procedure, the mixture of tibolone and 7α-methylnorethisterone is separated by techniques known to the skilled in the art, such as by chromatography, by crystallization or by a combination of these techniques.

It has been found that the conditions for the hydrolysis of the ketals of formula 2 put forth in the present invention ensure high selectivity toward tibolone as the major reaction product. Typically, tibolone is obtained in a large molar excess compared to 7α-methylnorethindrone, equal at least 2:1, more preferably 4:1, even more preferably 8:1.

The yield of tibolone obtained by this procedure is at least about 50% based on the 5(10)-estrene derivative of formula 2 and typically the yield of tibolone is much better. The amount of the side product formed is up to 20% based on the 5(10)-estrene derivative of formula 2, and typically it is much less.

According to the process of the present invention, 7α-methylnorethisterone of formula 3 can be conveniently reacted with a vicinal diol to form the 5(10)-estrene 3,3-ketal derivative of formula 2, which can be used again (recycled) in the hydrolysis step according to the present invention, leading to tibolone.

The reaction of 7α-methylnorethisterone of formula 3 with a diol is carried out, according to the process of the present invention, in the presence of an acid, preferably in the presence of a protic acid of pKa<1.5, most preferably in the presence of p-toluenesulfonic acid or an acid of a similar strength. Optionally, an organic non-polar solvent is used for the reaction, preferably toluene or xylenes. The reaction may optionally be carried out in the presence of a dehydrating agent, preferably a trialkyl orthoformate chosen from the group comprising trimethyl orthoformate, triethyl orthoformate, and/or triisopropyl orthoformate. According to the present invention, after the usual work-up procedure, the crude ketal of formula 2 is purified by techniques known to the skilled in the art, such as by chromatography, by crystallization or by a combination of these techniques. A preferred method of purification according to the present invention is by crystallization, more preferably by crystallization from a mixture of solvents containing 0%-50% THF, 0%-50% 1,4-dioxane, 0%-50% toluene and 0%-100% of ethyl acetate, and most preferably by crystallization from ethyl acetate, which is now found to be particularly efficient in removing any positional alkene isomers from the 5(10)-alkene product.

The process of the present invention is, in its principle, appropriate for production of tibolone on a small plant scale or on a plant scale. The preparation of the new 5(10)-estrene 3,3-ketals of formula 2 and the new process for their hydrolysis according to the present invention allow for a reduction in the number of synthetic steps compared to the prior art regarding the synthesis of tibolone from commercially available steroids. The mild acidic conditions used for the hydrolysis reaction according to the present invention, are easy to apply and control. Moreover, the inconvenient Birch reduction step is eliminated. In addition, according to the process of the present invention, 7α-methylnorethisterone of formula 3 (which is also known to be a physiologically active compound) formed as a side product during the hydrolysis, can be reacted with a diol, which efficiently gives a 3,3-ketal of formula 2.

The purification of compounds of formula 2, is substantially facilitated by the finding of the present invention that the crystallization from ethyl acetate alone, or from ethyl acetate in mixtures with other solvents, is very efficient in recovering pure compounds of formula 2, and in eliminating any positional double bond isomers. Thus, according to the process of the present invention, 7α-methylnorethisterone is recycled by reaction with a vicinal diol resulting in a compound of formula 2, which is then applied as a substrate for the last, hydrolytic step of tibolone preparation. This improves the overall chemical yield of the process of the present invention and lowers the cost of tibolone synthesis.

### Definitions and Conventions

The definitions and explanations below are for the terms as used throughout this entire document including both the specifications and the claims.

### Definitions

TLC refers to thin-layer chromatography,

RP refers to reversed phase,

RT refers to room temperature (ca. 25° C.),

THF refers to tetrahydrofuran

Chromatography (column and flash chromatography) refers to purification/separation of compounds expressed as (support; eluent). It is understood that the appropriate fractions are pooled, concentrated and dried under vacuum to give the specified compound.

When mixtures of solvents are used, the ratios of solvents used are volume/volume (v/v).

NMR refers to nuclear magnetic resonance spectroscopy, chemical shifts are reported in ppm (δ) downfield from tetramethylsilane.

### EXAMPLES

It is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### Preparation of 3,3-ethylenedioxy-7α-methyl-5(10)-estren-17-one (Formula 4, R₁-R₄=H)

Anhydrous NaOAc (analytical grade; 12.2 g), pyridinium chlorochromate (47.0 g, 218 mmol) and anhydrous CH₂Cl₂ (700 mL) were placed in a 2 liter flask. The mixture was stirred under nitrogen and cooled to 0° C. A solution of 3,3-ethylenedioxy-17-hydroxy-7α-methyl-5(10)-estrene (36.1 g, 108.6 mmol) in anhydrous CH₂Cl₂ (200 mL) was then added over 10 min. The mixture was stirred for 1 hr. Isopropanol (analytical grade, 6.0 mL) was then added and the mixture was stirred for 10 min., after which Et₂O (1.0 L) was added. After stirring for another 10 min., the mixture was filtered, the residue was washed with ether (3×150 mL), the filtrates were combined, anhydrous pyridine (1 mL) was added and the mixture was left at room temperature for 2 hrs. Afterwards, it was extracted with 10% aqueous KHCO₃ (2×3.00 mL) and dried over anhydrous Na₂SO₄ (280 g). The drying agent was filtered, then washed with CH₂Cl₂ (150 mL). The filtrates were combined, concentrated and dried under vacuum. This gave a pale yellow, glassy solid (35 g), which was additionally purified on a short flash column packed with silica gel (230-400 mesh, 0.4 kg; 15% EtOAc/hexane). The elution of the column with 20% EtOAc/hexane afforded 3,3-ethylenedioxy-7α-methyl-5(10)-estren-17-one as a colorless, glassy solid (29.0 g; 80.8%), which crystallized from diisopropyl ether (155 mL) to give 3,3-ethylenedioxy-7α-methyl-5(10)-estren-17-one of analytical purity (16.81 g); colorless crystals, m.p.: 141.5-143.8° C.; [α]_{D}=+160.50 (28° C., c=1, CHCl₃); ¹H-NMR (CDCl₃) δ 3.98 (4H, m), 2.47 (1H, m), 0.87 (3H, s, 18-Me.), 0.83 (3H, d: 7.1 Hz, 7α-Me); ¹³C-NMR (CDCl₃) δ 220.9, 128.0, 124.0, 108.2, 64.5, 64.2, 48.3, 47.3, 41.0, 40.5, 40.1, 38.4, 35.8, 31.9, 31.3, 26.7, 26.2, 24.7, 20.9, 14.0, 13.0.

### Example 2

### Preparation of 3,3-ethylenedioxy-17α-ethynyl-17β-hydroxy-7α-methyl-5(10)-estrene (Formula 2, R₁-R₄=H)

Potassium t-butoxide (71 g, 0.633 mol) was placed under nitrogen in a three-necked 1 liter flask equipped with a thermometer, a reflux condenser and a pipette-like inlet for acetylene. Anhydrous THF (550 mL) was added and the mixture was stirred at room temperature for 5 min., then the flask was immersed in an ice-water bath, the mixture was cooled to 0° C. and, with vigorous stirring, a gentle stream of acetylene was introduced. During the addition of acetylene the temperature rose to +8° C. and remained at this level for 2 hrs, after which time it dropped below +4° C. At this moment, the stream of acetylene was cut off, and a solution of 3,3-ethylene-dioxy-7α-methyl-5(10)-estren-17-one (28.6 g; 86.5 mmol) in anhydrous THF (150 mL) was added with vigorous stirring. The introduction of acetylene was then resumed. The mixture was vigorously stirred and cooled such that the temperature was maintained in the range +4 to +80° C. After 4 hrs, the mixture was cautiously transferred over 20 min. to a 6 liter reactor, containing a mixture of saturated NH₄Cl/H₂O (2.0 L) and toluene (1.0 L), which was vigorously stirred under nitrogen and cooled to 0° C. After 45 min. of stirring, the reactor was set aside for 1 hr at RT. The phases were then separated and the organic phase was dried over anhydrous Na₂SO₄ (300 g). The drying agent was filtered and washed with EtOAc (200 mL), the filtrates were combined and concentrated in vacuo. This latter operation was facilitated by the addition of ca. 15% v/v anhydrous THF to prevent spontaneous crystallization, which was causing foaming. The product was dried under vacuum and crystallized from hot ethyl acetate (100 mL; cooled to RT and left for 14 hrs) to give pure 3,3-ethylenedioxy-17-ethynyl-17-hydroxy-7a-methyl-5(10)-estrene (16.66 g, 54%); m.p.: 181-183° C.; [α]_{D}=+46.80° (28° C., c=1, CHCl₃); ¹H-NMR (CDCl₃) δ 3.98 (4H, m), 2.58 (1H, s), 0.85 (3H, s, 18-Me), 0.79 (3H, d: 7.1 Hz, 7α-Me); ¹³C-NMR (CDCl₃) δ 128.2, 123.7, 108.3, 87.7, 79.7, 73.7, 64.4, 64.1, 47.4, 46.2, 41.4, 41.0, 39.8, 38.9, 38.5, 33.1, 31.4, 27.2, 26.2, 25.1, 22.0, 13.0, 12.9.

### Example 3

### Preparation of 17α-ethynyl-17β-hydroxy-7α-methyl-5(10)-estren-3-one (Tibolone, Formula 1)

3,3-Ethylenedioxy-17α-ethynyl-17β-hydroxy-7α-methyl-5(10)-estrene (16.2 g, 45.4 mmol) was dissolved in anhydrous THF (100 mL). The solution was stirred at 40° C. under nitrogen, and ethanol (99.8 %; 500 mL) and water (140 mL) were added, followed by 96% formic acid (10.0 mL). After the mixture was stirred at 60° C. for 1 hr, methanol (100 mL) and formic acid (5.0 mL) were added and stirring under nitrogen was continued. The reaction was monitored on C-18 RP TLC plates developed with 10% H₂O/MeOH. After 6 hrs the reaction mixture was poured on a mixture of water (1.5 L) and pyridine (50 mL), which was stirred and cooled under nitrogen at +15° C. After 15 min. more water (0.5 L) was added and stirring was continued for another 30 min. The mixture was left at +4° C. for 14 hrs. The precipitate was filtered, dissolved in CH₂Cl₂ (300 mL) and extracted with 5% aqueous KHCO₃ (200 mL)). The phases were separated, the organic phase was dried over anhydrous Na₂SO₄ (50 g), filtered, concentrated and dried in vacuo. This gave a white solid (14.0 g) which was separated using flash chromatography and crystallization. Chromatography was performed on a column packed with silica gel (300 g, 230-400 mesh; 20% EtOAc—20% CH₂Cl₂—60% hexane). Crystallization was carried out from hot ethanol by slowly cooling the solution to RT and leaving it at this temperature for a day. This procedure afforded:
(a) 17α-ethynyl-17β-hydroxy-7α-methyl-5(10)-estren-3-one (tibolone; 9.04 g, 63.7%) as a white, crystalline powder; m.p.=165.8-168.8° C.; [α]_{D}=+103.2° (28° C., c=1, EtOH); HPLC purity of the sample reported herein was determined on a C-18 column using a standardized procedure: Rₜ=8.42 min, purity=99.12%; ¹H-NMR (CDCl₃; 200 MHz) δ 2.73 (2H, m), 2.59 (1H, s), 0.88 (3H, s, 18-Me) and 0.84 (3H, d: 7.0 Hz, 7α-Me)—spectrum in complete agreement with the spectrum obtained for a tibolone standard; ¹³C-NMR (CDCl₃; 50 MHz) δ 211.4, 129.8, 124.5, 87.6, 79.6, 73.8, 47.4, 46.0, 44.9, 41.7, 39.5, 39.1, 38.9, 38.4, 33.0, 27.4, 27.1, 25.2, 22.0, 13.0, and 12.8, and
(b) 17α-ethynyl-17β-hydroxy-7α-methyl-4-estren-3-one (formula 3; 2.70 g, 19.0%) as colorless prisms; m.p.: 200.5-202.5° C.; [α]_{D}=(−)24° (20° C., c=1, CHCl₃); UV λₘₐₓ=241 nm; ¹H-NMR (200 MHz; CDCl₃) δ 5.83 (1H, s), 2.57 (1H, s), 0.91 (3H, s) and 0.78 (3H, d: 7.0 Hz) ppm; ¹³C-NMR (50 MHz; CDCl₃) δ 199.6, 165.0, 126.5, 87.5, 79.5, 74.0, 46.9, 45.9, 43.5, 43.3, 43.0, 42.0, 38.8, 36.6, 32.3, 30.7, 26.7 (2C), 22.2, 12.8 and 12.6 ppm.

### Example 4

### Preparation of 17α-ethynyl-17β-hydroxy-7α-methyl-5 (10)-estren-3-one (Tibolone, Formula 1).

3,3-Ethylenedioxy-17α-ethynyl-17β-hydroxy-7α-methyl-5(10)-estrene (441 mg, 1.24 mmol) and anhydrous ethanol (10 mL) were stirred under nitrogen at 75° C. When the mixture became clear, methanol (5 mL) was added, quickly followed by a solution of CuSO₄×5 H₂O (320 mg, 1.28 mmol) in water (2 mL). The mixture was stirred under nitrogen while the heating bath temperature was maintained in the range of 73-76° C. The progress of the reaction was monitored by RP-TLC (C-18; 10% H₂O in MeOH). After 4.5 hrs more CuSO₄ ×5 H₂O (51 mg) was added and stirring was continued for another 0.5 hr. The reaction mixture was then cooled to +40° C. and, with vigorous stirring, 3% aqueous NaHCO₃ (70 mL) and CH₂Cl₂ (70 mL) were added. After extraction, the phases were separated and the aqueous phase was washed with CH₂Cl₂ (20 mL). The phases were separated, the organic phases were combined, dried over Na₂SO₄ and concentrated under vacuum. The products were isolated on a flash column packed with silica gel (30 g, 230-400 mesh; 20% EtOAc—10% CH₂Cl₂—70% hexane). The fractions homogenous on TLC were pooled, concentrated under vacuum and dried to a constant mass under vacuum. This gave:
(a) 17α-ethynyl-17β-hydroxy-7α-methyl-5(10)-estren-3-one (tibolone; 190 mg, 49%) as a white, crystalline powder; ¹H-NMR spectrum (200 MHz; CDCl₃) identical with a spectrum obtained for a tibolone standard, and
(b) 17α-ethynyl-17β-hydroxy-7α-methyl-4-estren-3-one (formula 3; 22 mg, 5.7%) as a white precipitate; ¹H-NMR spectrum (200 MHz; CDCl₃) identical with a spectrum obtained for an authentic sample of 17α-ethynyl-17β-hydroxy-7α-methyl-4-estren-3-one.

### Example 5

### Preparation of 3,3-ethylenedioxy-17α-ethynyl-17β-hydroxy-7α-methyl-5(10)-estrene (Formula 2, R₁-R₄=H) from 17α-ethynyl-17β-hydroxy-7α-methyl-4-estren-3-one

17α-Ethynyl-17β-hydroxy-7α-methyl-4-estren-3-one (3.46 g, 11 mmol) and anhydrous toluene (100 mL) were stirred under nitrogen at 65° C. Anhydrous ethylene glycol (12 mL) was added, followed by p-toluenesulfonic acid monohydrate (0.20 g). The mixture was vigorously stirred for 2 min., and anhydrous triethyl orthoformate (3.50 mL) was then added. The mixture was stirred under nitrogen at exactly 63-65° C., over 55 min. Powdered NaHCO₃ was then added in a few portions (total 2.20 g), the mixture was stirred for 5 min. and anhydrous pyridine (0.50 mL) was added. To prevent a loss of material caused by crystallization during work-up, THF (25 mL) was added. The mixture was cooled to +50° C., diluted with EtOAc (100 mL) and twice extracted with 10% aqueous KHCO₃ (2×150 mL). The phases were separated, the organic phase was diluted with THF (20 mL), the mixture was dried over Na₂SO₄, filtered and concentrated in vacuo to dryness. The crude product (4.1 g) was crystallized from hot EtOAc (30 mL). The crystallizing solution was left at RT for 20 hrs, then filtered to give 3,3-ethylenedioxy-17α-ethynyl-17β-hydroxy-7α-methyl-5(10)-estrene (2.46 g, 62%); identical by ¹H- and ¹³C-NMR with an authentic sample of pure 3,3-ethylenedioxy-17α-ethynyl-17β-hydroxy-7α-methyl-5(10)-estrene.

Although embodiments and examples of the invention have been shown and described, it is to be understood that various modifications, substitutions, and rearrangements of process steps, compounds and elements, as well as other methods for preparing the compounds of the invention can be made by those skilled in the art without departing from the novel spirit and scope of the invention.

What is claimed:

1. A process for the preparation of 17β-hydroxy-7α-methyl-19-nor-17α-pregn-5(10)-en-20-yn-3-one of formula 1, which comprises hydrolyzing 17α-ethynyl-17β-hydroxy-7α-methyl-5(10)-estrene 3,3-cyclic ketal of formula 2, where:
(1) each of R₁, R₂, R₃ and R₄ is a hydrogen atom or a C₁₋₄ alkyl group, or
(2) R₁ and R₃ are taken together to form an alicyclic ring together with the carbon atoms in the dioxolane ring to which the groups are attached and R₂, R₄ are hydrogen atoms, or
(3) R₁ and R₃ are taken together to form an aromatic ring together with the carbon atoms in the dioxolane ring to which they are attached, and R₂, R₄ are taken together to form a chemical bond participating in the aromatic electron system of the aromatic ring formed by R₁ and R₃.

2. A process according to claim 1, which comprises hydrolyzing 3,3-ethylenedioxy-17α-ethynyl-17β-hydroxy-7α-methyl-5(10)-estrene.

3. A process according to claim 1, characterized in that 17β-hydroxy-7α-methyl-19-nor-17α-pregn-5(10)-en-20-yn-3-one is obtained in a molar excess to 17α-ethynyl-17β-hydroxy-7α-methyl-4-estren-3-one, equal at least 2:1.

4. A process according to claim 3, characterized in that 17β-hydroxy-7α-methyl-19-nor-17α-pregn-5(10)-en-20-yn-3-one is obtained in a molar excess to 17α-ethynyl-17β-hydroxy-7α-methyl-4-estren-3-one, equal at least 4:1.

5. A process according to claim 4, characterized in that 17β-hydroxy-7α-methyl-19-nor-17α-pregn-5(10)-en-20-yn-3-one is obtained in a molar excess to 17α-ethynyl-17β-hydroxy-7α-methyl-4-estren-3-one, equal at least 8:1.

6. A process according to claim 1, where the hydrolysis reaction is carried out in a mixture of an organic solvent and water, in the presence of an acid having pKa value in water in the range of between 2 and 5 (pKa/_{H2O}=2-5).

7. A process according to claim 1, where the acid is chosen from a group comprising oxalic acid, acetic acid, fumaric acid, formic acid, malonic acid and pyridinium p-toluenesulfonate.

8. A process according to claim 7, where the acid is formic acid.

9. A process according to claim 1, characterized in that the hydrolysis reaction is carried out in a mixture containing an organic solvent and water, in the presence of salts of transition metals.

10. A process according to claim 1, characterized in that the hydrolysis reaction is carried out in a mixture containing an organic solvent and water, in the presence of lithium or magnesium.

11. A process according to claim 9, where the salt is copper(II) sulfate.

12. A process according to claim 1, characterized in that the hydrolysis reaction is carried out in a mixture of solvents containing 0%-99% water, 0%-100% of a co-solvent selected from a group consisting of THF, CHCl₃, 1,4-dioxane, CH₂Cl₂, acetone, acetonitrile, ethylmethylketone, diethylketone, 1,3-dioxolane, 1,2-dimethoxyethane, 1,2-diethoxyethane, and 0%-100% of a C₁₋₄ alcohol.

13. A process according to claim 1, where the reaction temperature is from about 0° C. to about 200° C.

14. A process according to claim 1, characterized in that 17α-ethynyl-17β-hydroxy-7α-methyl-4-estren-3-one of formula 3 is converted to a ketal of formula 2, which is then hydrolyzed to 17β-hydroxy-7α-methyl-19-nor-17α-pregn-5(10)-en-20-yn-3-one.

15. A process according to claim 14, characterized in that 17α-ethynyl-17β-hydroxy-7α-methyl-4-estren-3-one of formula 3 is converted to a 17α-ethynyl-17β-hydroxy-7α-methyl-5(10)-estrene 3,3-ketal of formula 2 by reaction with a vicinal diol, in the presence of a protic acid and a hydrocarbon solvent.

16. A process according to claim 15, characterized in that the 17α-ethynyl-17β-hydroxy-7α-methyl-5(10)-estrene 3,3-ketal of formula 2 is purified before the hydrolysis step, by crystallization from a mixture of solvents containing 0%-50% THF, 0%-50% 1,4-dioxane, 0%-50% toluene and 0%-100% of ethyl acetate, preferably by crystallization from ethyl acetate.

17. A composition, comprising: 17α-ethynyl-17β-hydroxy-7α-methyl-5(10)-estrene 3,3-cyclic ketal of formula 2, where
(1) each of R₁, R₂, R₃ and R₄ is a hydrogen atom or a C₁₋₄ alkyl group, or
(2) R₁ and R₃ are taken together to form an alicyclic ring together with the carbon atoms in the dioxolane ring to which the groups are attached and R₂, R₄ are hydrogen atoms, or
(3) R₁ and R₃ are taken together to form an aromatic ring together with the carbon atoms in the dioxolane ring to which they are attached, and R₂, R₄ are taken together to form a chemical bond participating in the aromatic electron system of the aromatic ring formed by R₁ and R₃.

18. A compound, comprising: 17α-ethynyl-17β-hydroxy-7α-methyl-5(10)-estrene 3,3-cyclic ketal of formula 2, where R₁, R₂, R₃, and R₄ are hydrogen atoms.

19. A compound according to claim 18, of about 90% purity.

20. A compound according to claim 18, of purity better than 90%.

21. A process for the preparation of pure 17α-ethynyl-17β-hydroxy-7α-methyl-5(10)-estrene 3,3-cyclic ketals of formula 2, where R₁-R₄ are hydrogen atoms, characterized in that 7α-methyl-5(10)-estren-17-one 3,3-cyclic ketal of formula 4, where R₁-R₄ are hydrogen atoms, is reacted with a metal acetylide, in an inert solvent, while maintaining the temperature of the reaction mixture in the range from about −50° C. to about +30° C.

22. A process according to claim 21, characterized in that, prior to the addition reaction, said metal acetylide is generated from acetylene gas, in the same reaction pot in which the addition to 7α-methyl-5(10)-estren-17-one 3,3-cyclic ketal of formula 4 will subsequently be carried out.

23. A process according to claim 22, characterized in that the said reaction product of formula 2 is further purified by crystallization from a solvent containing 50-100% ethyl acetate.

24. A process according to claim 6, where the acid is chosen from a group comprising oxalic acid, acetic acid, fumaric acid, formic acid, malonic acid and pyridinium p-toluenesulfonate.
